(19) United States
(12) Patent Application Publication
(10) Pub. No.: US 2008/0206379 A1
(43) Pub. Date: Aug. 28, 2008

(54) Production and Use of an Antioxidant Extract from Crypthecodinium Sp.

(76) Inventors: Dirk Fabritius, Ansbach (DE); Doreen Neumann, Hattersheim (DE)

Correspondence Address: HAMMER & ASSOCIATES, P.C., 3125 SPRINGBANK LANE, SUITE G, CHARLOTTE, NC, 28226, US

(21) Appl. No.: 11/814,307

(22) PCT Filed: Jan. 26, 2006
(86) PCT No.: PCT/EP2006/000676

§ 371 Date: Aug. 7, 2007

(30) Foreign Application Priority Data
Jan. 26, 2005 (DE) 10 5005 003 624.4

(51) Int. Cl. A61K 35/66; C11B 5/00; A61P 35/00

(52) U.S. Cl. 424/780; 426/542

(57) ABSTRACT

The invention relates to an antioxidant extract from Crypthecodinium sp. The invention also relates to a fatty acid composition which comprises at least one unsaturated fatty acid and/or at least one unsaturated fatty acid ester and at least one extract according to the invention and constituents of a biomass different from Crypthecodinium sp., to a method for producing the fatty acid composition and to its use.

The present invention relates to an extract from Crypthecodinium sp., a method for its production and also its use, in particular for antioxidative stabilization of fatty acid compositions which contain one or more long-chain polyunsaturated fatty acids and/or one or more long-chain polyunsaturated fatty acid esters.

Long-chain polyunsaturated fatty acids (PUFAs) are essential fatty acids in human metabolism. PUFAs can be subdivided into two large groups. In addition to the group of ω-6 PUFAs, which are formulated proceeding from linoleic acid, there is the group of ω-3 PUFAs which are made up starting from α-linolenic acid.

PUFAs are important building blocks of cell membranes, the retina and the meninges and precursors of important hormones, for example prostaglandins, thromboxanes and leukotrienes.

In addition to the function as building blocks, in the course of recent years it has increasingly been found that PUFAs directly have multiple beneficial effects on the human organism or diseases.

A multiplicity of clinical studies have found that PUFAs can make an important contribution to healing or alleviation, for example in the case of cancer, rheumatoid arthritis, high blood pressure and neurodermatitis and many other diseases. In these cases the use of docosahexaenoic acid (DHA; all-cis-4,7,10,13,16,19-docosahexaenoic acid) and their derivatives, in particular DHA esters, is frequently particularly advantageous, because such esters (in particular the ethyl esters and triglycerides) have a tendency to have a pleasant taste and to be readily absorbed by the digestive system. These findings were originally responsible for the fact that international institutions and authorities have delivered recommendations which control the daily intake of PUFAs.

PUFAs cannot be synthesized de-novo by humans, since they lack the enzyme systems which can introduce a double bond into the carbon chain at positions >C9 (lack of Δ12-desaturase). Humans are only able to synthesize polyunsaturated fatty acids via the supply of what are termed precursor fatty acids (for example α-linolenic acid) from the diet. However, whether this amount is sufficient to cover the requirement of polyunsaturated fatty acids is contested.

The great majority of essential fatty acids are taken in via the diet. In particular vegetable oils are enriched with ω-6 fatty acids (for example evening primrose oil contains γ-linolenic acid (GLA)) but only up to a chain length of C18, and fish oils and oils from microorganisms, with ω-3 fatty acids (for example salmon oil contains eicosapentaenoic acid (EPA) and docosahexaenoic acid (DHA; all-cis-4,7,10,13,16,19-docosahexaenoic acid)). In principle, fish oils and oils from microorganisms are the only commercial source of polyunsaturated fatty acids. Generally, however, the content of the desired PUFAs is too low and they are present in a mixture, in which case PUFAs acting antagonistically can also be present. In order to consume the recommended daily dose of PUFAs, therefore, a high quantity of oil must be consumed. In particular, this applies to those patients who must consume high doses of PUFAs (for example in the case of cystic fibrosis). To achieve an effect of the individual PUFAs in as targeted manner as possible, enriched or high-purity PUFAs must be used. Therefore, in the prior art, there is a great requirement for high-purity PUFAs.

Numerous methods have been used individually or in combination to isolate (or at least concentrate) and recover certain fatty acids and their derivatives from a multiplicity of naturally occurring sources. These methods include fractional crystallization at low temperatures, molecular distillation, urea adduct crystallization, extraction with metal salt solutions, supercritical fluid fractionation on countercurrent columns and HPLC methods.

On account of their sensitivity to oxidation, PUFAs must generally be stabilized by adding suitable antioxidants. Commercially, for this purpose, use is especially made of natural tocopherols, in particular mixtures of α-, β-, γ-, δ-tocopherol and/or tocotrienols extracted from soybean oil. In addition, it is known that some compounds such as, for example, ascorbyl palmitate, can act synergistically. They are therefore used in addition to the tocopherol.

The effect of natural antioxidants, however, does not increase in an unlimited manner with increasing concentration. For example, in the case of α-tocopherol, the activity reverses as early as at 100 ppm, and a pro-oxidant activity occurs. This means that overdosing can also have adverse consequences.

Alternatively, the publication WO03092628 proposes the use of an oil worked up under mild conditions. The preparation must proceed in this case in such a manner that a polyunsaturated fatty acid-containing biomass is first reacted with an enzyme and the lipid is subsequently isolated. Although the oil obtainable in this manner is at first apparently not so greatly oxidized, it nevertheless exhibits the sensitivity to oxidation characteristic of polyunsaturated fatty acids.

In the light of this prior art, it was therefore an object of the present invention to indicate possibilities for enhanced antioxidative stabilization of fatty acid compositions. Increasing the antioxidant activity in this case should be achieved as far as possible without adding substances hazardous to health in order to enable applications of the fatty acid composition in the food sector without reservations.

A further object of the present invention was specifying a method for producing the fatty acid composition of the invention which permits its production in as simple a manner as possible on a large scale and inexpensively.

Furthermore, particularly advantageous fields of application of the fatty acid composition according to the invention should be indicated. These and other objects which, although they are not mentioned explicitly, may be derived as obvious from the contexts discussed herein or inevitably result from these, are achieved by an antioxidant extract from Crypthecodinium sp.

Expedient modifications of the extract according to the invention are described in the subclaims which are referred back to claim 1. Claims 11 to 19 are antioxidant-stabilized fatty acid compositions under the scope of protection. The method claim protects a particularly suitable mode of production of the fatty acid composition according to the invention and the use claims describe particularly advantageous fields of application of the fatty acid composition according to the invention.

By providing an antioxidant extract from Crypthecodinium sp., an extract having particularly high antioxidant activity is successfully made accessible, in a manner not readily foreseeable, which extract is suitable in particular for the antioxidative stabilization of fatty acid compositions, especially those fatty acid compositions which contain at least one unsaturated fatty acid and/or at least one unsaturated fatty acid ester. In this case the increase in antioxidant activity is achieved according to the invention without addition of substances hazardous to health, that is to say use of the fatty acid composition according to the invention is possible in the food sector without concern. For instance, Crypthecodinium cohnii oil is already used in infant feeding and is categorized in the USA as GRAS (Generally Recognized As Safe).

The fatty acid composition according to the invention can be produced in a simple manner, on a large scale and inexpensively.

The fatty acid composition contains according to the present invention at least one antioxidant extract from Crypthecodinium sp., preferably from Crypthecodinium cohnii. The expression “fatty acid composition” in this context comprises not only compositions which contain free fatty acids, but also compositions which fatty acid derivatives, preferably fatty acid esters, in particular fatty acid triglycerides, in which case the fatty acid radicals can in principle be identical or different.

Fatty acids denote according to the invention aliphatic carboxylic acids which can be saturated or monounsaturated or polyunsaturated and preferably have 6 to 30 carbon atoms.

Extracts obtainable from Crypthecodinium sp. are known per se. According to the invention, use can be made not only of extracts of Crypthecodinium sp. wildtype strains, but also extracts of mutant or recombinant Crypthecodinium sp. strains.

The expression “extract from Crypthecodinium sp.” in the present context comprises all compositions which can be obtained by extraction of a biomass, preferably an oil, of Crypthecodinium sp. with a solvent, preferably with an organic and/or supercritical solvent, in particular with an organic solvent. The use of solvent mixtures is likewise possible.

According to the invention the extract has an antioxidant activity which is preferably greater than that of the biomass from which the extract is obtained. It therefore preferably has a peroxide value which is less than the peroxide value of the originally used, preferably freshly isolated, biomass from which the extract is obtained, and is preferably at most 50.0%, more preferably at most 25.0%, expediently at most 10.0%, in particular at most 1.0%, of the peroxide value of the biomass from which the extract is obtained. The peroxide value in this case is preferably determined as specified in AOCS Official Method Cd-3d 63 (American Oil Chemists Society), expediently after open storage for 2 weeks.

The antioxidative capacity of the extract according to the invention is preferably greater than 15 000 Trolox equivalents, more preferably greater than 20 000 Trolox equivalents, expediently greater than 25 000 Trolox equivalents, particularly preferably greater than 30 000 Trolox equivalents, and in particular greater than 35 000 Trolox equivalents (μg/ml). Trolox® is the customarily used trade name of 6-hydroxy-2,5,7,8-tetramethylchromane-2-carboxylic acid.

The expression “biomass of an organism” comprises according to the invention not only whole cells of the organism but also individual cell components of the organism.

The extract from Crypthecodinium sp. is expediently obtained by culturing the microorganism, harvesting the biomass from the culture, disintegrating it and isolating the extract.

For isolation of the extract, preferably use is made of extraction methods with organic solvents, in particular hexane, or with supercritical liquids. Extraction methods with organic solvents are particularly preferred in this case. Expediently, the extract is extracted from the biomass by percolation of the dried biomass with hexane. Such extractions with organic solvents are described, inter alia, in WO 9737032, in WO 9743362 and EP 515460. A particularly extensive description may also be found in Journal of Dispersion Science and Technology, 10, 561-579, 1989 “Biotechnological Processes for the Production of PUFAs”.

Alternatively, the extraction can also proceed without solvent. A particularly expedient method in this context is described in EP-A-1178118. In this method a solvent is avoided by producing an aqueous suspension of the biomass and separating off the oil phase from the aqueous phase by centrifugation.

According to a particularly preferred variant of the present invention, the extract is obtained by pure mechanical pressing of a biomass from Crypthecodinium sp. and subsequent extraction with at least one organic solvent or at least one supercritical solvent, preferably with an organic solvent, in particular with hexane.

According to a further particularly preferred variant of the present invention, the extract is obtained by distillation.

In the context of the present invention it has also proved to be very particularly advantageous to transesterify the biomass, preferably with an aliphatic alcohol having 1 to 12 carbon atoms, preferably having 1 to 6 carbon atoms, in particular having 1 to 4 carbon atoms. In this case the use of methanol and ethanol, in particular of ethanol, is very particularly proven. The transesterification preferably proceeds under acid catalysis, in particular with the use of sulfuric acid and/or hydrochloric acid. According to a further particularly preferred variant, the transesterification is achieved enzymatically.

The transesterified biomass is subsequently preferably extracted with at least one organic solvent or supercritical solvent, preferably with an organic solvent, in particular with hexane. The ratio of the total volume of the solvent to the volume of the reaction mass (including the added water) can also be varied within a broad range and is particularly preferably from 1:3 to 4:3. According to a particularly preferred embodiment, the mixture is extracted with a plurality of parts of the solvent which are combined at the end.

In the context of this embodiment, preferably use is made of a hexane extract of a biomass of Crypthecodinium sp. as biomass to be transesterified which is then transesterified as described above. This process serves for concentration and purification of the antioxidant extract. Expediently, the extract which is concentrated and purified in this manner, based on its total weight, has a content of fatty acids having 6 to 30 carbon atoms and of fatty acid esters which comprise fatty acid alkyl radicals having 6 to 30 carbon atoms of less than 20.0% by weight, preferably of less than 10.0% by weight, in particular of less than 5.0% by weight.

The composition of the extract can vary within a broad range. In the context of a first particularly preferred embodiment of the present invention, the extract from Crypthecodinium sp. is obtainable by
i) saponifying a biomass of Crypthecodinium sp. and
ii) extracting the saponified biomass with a solvent which has a water solubility less than 0.1 g of solvent per g of water at 25° C.

Preferably, in this case, the procedure of the DGF method F-II 1 (75) is followed.

The biomass can be saponified in a manner known per se. In this case reaction of the biomass with at least one alkali metal hydroxide, preferably with NaOH and/or KOH, in particular with KOH, in alcoholic solution, preferably in methanolic and/or ethanolic solution, is particularly proven. Particularly suitable reaction temperatures for the saponification are in the range from 25 to 100° C.

Extraction of the saponified product mixture can vary within a wide range. According to a preferred variant, water is added to the mixture and extraction is performed with a solvent which has a water solubility less than 0.1 g of solvent per g of water at 25° C. The ratio of the total volume of the solvent to the volume of the reaction mass (including the added water) can also be varied within a wide range and is particularly preferably from 1:3 to 4:3. According to a particularly preferred embodiment, the mixture is extracted with a plurality of parts of the solvent which are combined at the end. Solvents which are particularly suitable according to the invention include the organic solvents dichloromethane, diethyl ether, methyl ethyl ketone, ethyl acetate, petroleum ether, pentane and hexane and also the supercritical solvents propane, butane and carbon dioxide, with the organic solvents, especially diethyl ether and hexane, in particular diethyl ether, being most preferred.

Remaining water can be removed from the extraction solvent layer by, for example, washing the layer with a brine (that is to say a saturated salt solution), by drying with a molecular sieve and/or by drying with an anhydrous salt (for example sodium sulfate or magnesium sulfate).

After the extraction, the extract is preferably concentrated, expediently by partially or completely evaporating the solvent.

In the context of a further particularly preferred embodiment of the present invention, the extract from Crypthecodinium sp. is obtainable by extraction of a biomass of Crypthecodinium sp. with an alcohol having 1 to 12, preferably 1 to 6, in particular 1 to 4, carbon atoms and/or with a ketone having 3 to 6, preferably 3 or 4, carbon atoms. The extraction with an alcohol in this case is preferred to extraction with a ketone. Alcohols which are very particularly suitable for the present purposes, in each case, individually or in a mixture, are methanol and ethanol.

Particularly suitable ketones comprise acetone and/or methyl ethyl ketone, in particular acetone.

In the context of this embodiment, use is preferably made of a hexane extract of a biomass of Crypthecodinium sp. as biomass to be extracted which is then counterextracted with the alcohol and/or ketone. This process serves for concentration and purification of the antioxidant extract. Expediently, the extract concentrated and purified in this manner, based on its total weight, has a content of fatty acids having 6 to 30 carbon atoms and fatty acid esters which comprise fatty acid radicals having 6 to 30 carbon atoms of less than 20.0% by weight, preferably less than 10.0% by weight, in particular less than 5.0% by weight.

The ratio of the total volume of alcohol or ketone to the volume of biomass can be varied in this case within a wide range and is particularly preferably from 3:1 to 3:4. According to a particularly preferred embodiment, the mixture is extracted with a plurality of parts of the alcohol or ketone which are combined at the end.

After the extraction, the extract is preferably concentrated, expediently by evaporating the solvent in part or completely.

The extract obtainable in this manner is preferably again extracted with a ketone having 3 to 6 carbon atoms, more preferably with acetone and/or methyl ethyl ketone, in particular with acetone. The ratio of the total volume of the ketone to the volume of the first extract can be varied within a wide range in this case and is particularly preferably from 3:1 to 3:4. According to a particularly preferred embodiment, the first extract is extracted with a plurality of parts of the ketone which are combined at the end.

After the extraction the resultant second extract is preferably concentrated, expediently by evaporating the solvent in part or completely.

In the context of the present invention, the fatty acid composition in addition contains components of a biomass different from Crypthecodinium sp., preferably a biomass of Thraustochytriales, in particular a biomass of Ulkenia sp. Biomasses different from Crypthecodinium sp. are likewise known per se. According to the invention, use can be made not only of biomasses of wildtype strains but also biomasses of mutant or recombinant strains which produce DHA (all-cis-4,7,10,13,16,19-docosahexaenoic acid) and/or DPA (all-cis-4,7,10,13,16-docosapentaenoic acid) efficiently. Such mutant or recombinant strains include microorganisms which, compared with the percentage of the original wildtype strain, using the same substrate, contain a higher percentage of DHA and/or DPA in fats, and/or compared with the amount produced by the original wildtype strain, using the same substrate, contain a higher total amount of lipids.

According to a particularly preferred embodiment of the present invention, the fatty acid composition according to the invention contains an extract of the biomass different from Crypthecodinium sp. The extract in this case is expediently obtained by culturing the microorganism in question, harvesting the biomass from the culture, disintegrating it and isolating the extract. A method which is very particularly expedient in this context is described in WO 03/033631 A1, the disclosure of which is hereby explicitly incorporated by reference.

For isolation of the extract, preferably use is made of extraction methods with organic solvents, in particular hexane, or with supercritical liquids. Expediently, the extract is extracted from the biomass by percolation of the dried biomass with hexane. Such extractions with organic solvents are described, inter alia, in WO 9737032, in WO 9743362 and EP 515460. A particularly extensive description may also be found in Journal of Dispersion Science and Technology, 10, 561-579, 1989 “Biotechnological Processes for the Production of PUFAs”.

Alternatively, the extraction can also proceed without solvent. A method which is particularly expedient in this context is described in EP-A-1178118. In this method a solvent is avoided by producing an aqueous suspension of the biomass and separating off the oil phase from the aqueous phase by centrifugation.

According to a particularly preferred variant of the present invention, the extract is obtained by pure mechanical pressing of a biomass different from Crypthecodinium sp. and subsequent extraction with at least one organic or supercritical solvent, preferably with at least one organic solvent, in particular with hexane.

In the context of the present invention it has proved to be particularly advantageous to transesterify the biomass, preferably with an aliphatic alcohol having 1 to 12 carbon atoms, preferably having 1 to 6 carbon atoms, in particular having 1 to 4 carbon atoms. In this case the use of methanol and ethanol, in particular ethanol, is very particularly proven. The transesterification preferably proceeds under acid catalysis, in particular with use of sulfuric acid and/or hydrochloric acid. The transesterified biomass is subsequently preferably extracted with an organic solvent, in particular with hexane. The ratio of the total volume of the solvent to the volume of the reaction mass (including the added water) can also be varied within a wide range and is particularly preferably from 1:3 to 4:3. According to a particularly preferred embodiment, the mixture is extracted with a plurality of parts of the solvent which are combined at the end.

The composition of the biomass can vary within a broad range. Preferably, the biomass different from Crypthecodinium sp. contains at least one polyunsaturated fatty acid and/or at least one fatty acid ester expediently one fatty acid alkyl ester, preferably a glyceride, in particular a triglyceride, which comprises at least one polyunsaturated fatty acid radical which preferably has 6 to 30 carbon atoms. According to a particularly preferred embodiment, at least 10%, particularly preferably at least 25%, and in particular at least 30%, of the fatty acids and/or the fatty acid radicals in the biomass are DHA or DHA radicals.

A “glyceride” is, as far as the expression is used herein, an ester of glycerol and at least one fatty acid, wherein one to three hydroxyl groups of the glycerol were esterified with one or more fatty acid radicals. When a plurality of fatty acid radicals are present, the fatty acid radicals can be identical or different.

In many suitable starting materials, the majority of the glycerides are triglycerides, that is to say esters of three fatty acid radicals and glycerol. In this case each fatty acid radical can either be saturated (that is to say all bonds between the carbon atoms are single bonds) or unsaturated (that is to say there is at least one carbon-carbon double bond or triple bond). The type of the unsaturated fatty acid radicals is sometimes designated herein by an ω. This number gives the position of the first double bond, counting starting from the terminal methyl group of the fatty acid or of the fatty acid radical.

The relative fractions of the individual components of the fatty acid composition according to the invention can in principle be chosen freely and matched to the respective use. In the context of the present invention, however, it has been found to be very particularly expedient when the fatty acid composition, in each case based on its total weight, contains 0.1 to 50.0% by weight, preferably 0.1 to 25.0% by weight, expediently 0.2 to 10.0% by weight, in particular 0.5 to 5.0% by weight, of the antioxidant extract from Crypthecodinium sp. and 50.0 to 99.9% by weight, preferably 75.0 to 99.9% by weight, expediently 90.0 to 99.8% by weight, in particular 95.0 to 99.5% by weight, components of a biomass different from Crypthecodinium sp., with the abovementioned relative fractions taken together preferably giving 100.0% by weight.

The fatty acid composition according to the invention has a relatively high fraction of polyunsaturated fatty acids and contains, in each case based on its total weight, preferably at least 10.0% by weight, expediently at least 25.0% by weight, more preferably at least 50.0% by weight, in particular at least 70.0% by weight, docosahexaenoic acid (all-cis-4,7,10,13,16,19-docosahexaenoic acid) and/or docosahexaenoic acid alkyl ester (all-cis-4,7,10,13,16,19-docosahexaenoic acid alkyl ester), preferably docosahexaenoic acid, docosahexaenoic acid methyl ester and/or docosahexaenoic acid ethyl ester.

The fatty acid composition according to the invention is distinguished, in comparison with conventionally stabilized fatty acid compositions, by a higher stability to oxidation. The addition of antioxidants which are known per se, such as, for example, α-, β-, γ- and/or δ-tocopherol, is therefore not absolutely necessary. Accordingly, the fatty acid composition according to the invention, according to a first preferred embodiment, does not contain further antioxidants.

However, since the antioxidative stability of the fatty acid composition according to the invention can frequently be further increased by the additional addition of antioxidants, the fatty acid composition according to a very particularly preferred embodiment of the invention contains at least one, preferably synergistically acting, antioxidant, preferably at least one tocotrienol, α-, β-, γ- and/or δ-tocopherol, expediently α-, β-, γ- and/or δ-tocopherol, in particular α-, β-, γ- and/or δ-tocopherol and ascorbyl palmitate, the relative fraction of this component preferably being 0.01 to 5.0% by weight, in particular 0.05 to 0.5% by weight, in each case based on the total weight of the fatty acid composition.

The fatty acid composition according to the invention is produced in a manner known per se, preferably by mixing the corresponding components. In this case it has proved to be very particularly advantageous to dissolve the antioxidant extract from Crypthecodinium sp. and the components of the biomass different from Crypthecodinium sp. separately from one another in a solvent, preferably petroleum ether, hexane, pentane, ethanol, methanol, acetonitrile, dichloromethane, methyl ethyl ketone, diethyl ether and/or ethyl acetate, expediently hexane and/or diethyl ether, in particular diethyl ether, then to mix the solutions with one another and subsequently to remove the solvent, preferably by evaporation.

According to a further preferred embodiment of the invention, the components are mixed without addition of solvent, in which case if appropriate elevated temperatures, preferably in the range from 25° C. to 80° C., in particular in the range from 25° C. to 60° C., are used.

Possible fields of application of the fatty acid composition according to the invention are immediately obvious to those skilled in the art. They are suitable, in particular, for all applications which are indicated for PUFAs and PUFA esters. In such cases the fatty acid composition according to the invention can usually be used directly. However, for some applications it is necessary to saponify in advance the fatty acid ester or the fatty acid esters in the liquid phase. This can be achieved, for example, by reaction with KOH in ethanol and subsequent acidification with an inorganic or organic acid.

The fatty acid composition according to the invention is used, in particular, as active ingredient or component in pharmaceutical compositions, as component in cosmetics preparations, as food additive, as food ingredient, as component of functional foods and for producing highly concentrated PUFA secondary products, such as esters and acids.

The invention will be described in more detail hereinafter by examples, without the inventive concept being hereby restricted.

The induction time, the peroxide values and/or the antioxidative capacity of the following fatty acid compositions were determined:

### Control 1

A “DHA-containing oil” produced as described in Yokochi et al., Appl. Microb. Biotechnol., (1998), 49, pp. 72-76, was used. This was subjected to complete refining by generally known method steps. Hereinafter this oil is designated as “DHA-containing oil” for short.

### Control 2-17

“DHA-containing oil”+the amounts of ascorbyl palmitate and/or tocopherol mixture (added 0.14% ®Coviox T70; natural tocopherol mixture) specified in table 1.

### EXAMPLE 1

The extract was obtained in accordance with DGF method F-II 1 (75).

5.02 g of Crypthecodinium cohnii crude oil (hexane extract) were weighed into a 250 ml round-bottom flask and admixed with 20 mg of pyrogallol, 40 ml of methanol, 10 ml of 60% strength potassium hydroxide solution (g/v) and 3 boiling chips. In an 80° C. hot water bath, the sample was saponified for 20 minutes under reflux and a gentle nitrogen stream. After cooling, the soap solution was flushed 3 times with 40 ml of twice distilled water and twice with 50 ml of diethyl ether into a 500 ml separating funnel.

A first extraction proceeded with the diethyl ether with careful swirling. The aqueous phase was let out into a 600 ml glass beaker. The diethyl ether phase was rewashed with 40 ml of twice distilled water, the water was drained to the aqueous phase. The diethyl ether phase was drained into a 1000 ml round-bottom flask. The aqueous phase was treated again four times as described (diethyl ether added, extraction etc.) until it was colorless. The combined diethyl phases were concentrated on a rotary evaporator, dried using an oil pump and weighed. This produced 921 mg of extract.

This was admixed with 4 g of “DHA-containing oil” (control 5; contains 0.1% tocopherol) and mixed well with the addition of 10 ml of diethyl ether. After removal of the diethyl ether, an orange oil was obtained.

### EXAMPLE 2

41.9 g of Crypthecodinium cohnii crude oil were weighed into a 500 ml round-bottom flask, admixed with 120 ml of methanol and a magnetic stirring bar. The batch was stirred vigorously for 3 hours on the magnetic stirrer. The upper methanol phase was decanted off into a 250 ml round-bottom flask. The oil batch was again admixed with 100 ml of methanol and rewashed for one hour. The oil-methanol mixture was placed into a 100 ml separating funnel and the methanol phase transferred to the previous one. This was concentrated on a rotary evaporator and dried by means of an oil pump. This produced 760 mg of extract. A “DHA-containing oil” (control 5; contains 0.1% tocopherol) was admixed with 2% by weight of the extract and mixed well.

### EXAMPLE 3

Obtained in a similar manner to the fatty acid composition from example 2, except that the “DHA-containing oil” (control 1) used was admixed with 4% by weight of the extract and mixed well.

### EXAMPLE 4

A Crypthecodinium cohnii dry biomass was extracted directly with methanol, in which case a large fraction of phospholipids was also co-extracted, which led to a very viscous product.

A “DHA-containing oil” (control 1) was admixed with 4% by weight of the extract and mixed well.

### EXAMPLE 5

A Crypthecodinium cohnii dry biomass was extracted directly with methanol, in which case a large fraction of phospholipids was also co-extracted, which led to a very viscous product. To remove these compounds the extract was again washed with acetone and the acetone-soluble components formed the Crypthecodinium cohnii acetone extract.

A “DHA-containing oil” (control 1) was admixed with 4% by weight of the acetone extract and mixed well.

### EXAMPLE 6

Obtained in a similar manner to the fatty acid composition from example 2, except that the “DHA-containing oil” (control 5) used was admixed with 4% by weight of the extract and mixed well.

Rancimat determination

| | |
|---|---|
| Instrument: | 743 Rancimat |
| Manufacturer: | Metrohm |
| Instrument settings: | |
| Method: | (similar to AOCS method Cd12b-92) |
| Temperature: | 80° C. |
| Gas flow rate: | 20 L/h |
| Stop criterion: | endpoint |

### Procedure and Principle of Measurement:

The oil (3 g) to be measured is weighed into a reaction vessel, placed into the heating block and exposed to a defined temperature and an air stream. Volatile oxidation products are formed, such as formic acid, which are transferred via an air tube into the measurement vessel in which the conductivity is measured in distilled water using the conductivity electrode. The conductivity is recorded over time to the endpoint. From this curve the second derivative is automatically formed which has its maximum at the saddle point. The time up to the saddle point is termed the induction time.

The higher the stability of the respective sample, the higher is also the induction time. Accordingly, by comparing the measured induction times, conclusions can be drawn as to the anti-/oxidative status of a sample and also the activity of antioxidants can be effectively compared with one another.

For the materials listed above, the induction times summarized in table 1 were measured.

TABLE 1
Induction times by the Rancimat test

| Sample | Addition | Induction time (h) |
|---|---|---|
| Control 1 | — | 1.1 |
| Control 2 | 0.01% by weight Toc | 1.9 |
| Control 3 | 0.025% by weight Toc | 3.7 |
| Control 4 | 0.05% by weight Toc | 5.5 |
| Control 5 | 0.1% by weight Toc | 5.7 |
| Control 6 | 0.15% by weight Toc | 6.8 |
| Control 7 | 0.2% by weight Toc | 7.7 |
| Control 8 | 0.5% by weight Toc | 7.0 |
| Control 9 | 1.0% by weight Toc | 7.5 |
| Control 10 | 2.0% by weight Toc | 6.6 |
| Control 11 | 0.025% by weight Toc + 0.025% by | 4.3 |
| | weight AP | |
| Control 12 | 0.1% by weight Toc + 0.025% | 9.0 |
| | by weight AP | |
| Control 13 | 0.1% by weight Toc + 0.5% | 8.5 |
| | by weight AP | |
| Control 14 | 0.1% by weight Toc + 0.1% | 7.4 |
| | by weight AP | |
| Control 15 | 0.2% by weight Toc + 0.05% | 11.6 |
| | by weight AP | |
| Control 16 | 0.2% by weight Toc + 0.1% | 10.6 |
| | by weight AP | |
| Control 17 | 0.2% by weight Toc + 0.2% | 7.0 |
| | by weight AP | |
| Example 1 | 18.7% by weight UVA + 0.1% | 17.9 |
| | by weight Toc | |
| Example 2 | 2.0% by weight MeOH-extr. + 0.1% | 14.3 |
| | by weight Toc | |
| Example 6 | 4.0% by weight MeOH-extr. + 0.1% | 17.6 |
| | by weight Toc | |
| Example 5 | 4% by weight Ace-extr. | 41.5 |

AP: ascorbyl palmitate
Toc: tocopherol mixture
uvA: unsaponifiable fractions (see above)
MeOH-extr.: MeOH extract (see above)
Ace-extr.—acetone extract (see above)

### Determination of Peroxide Values

The materials above were stored for predetermined times in open 100 ml Erlenmeyer flasks in the dark at room temperature and subsequently analyzed for their peroxide values. The peroxide values were determined as specified in AOCS Official Method Cd-3d 63 (American Oil Chemists Society). The results obtained are summarized in table 2. They show that by using the methanol extract (example 3) the antioxidative stability can be significantly increased compared with the “DHA-containing oil” without additional stabilizer (control 1) or the conventionally stabilized “DHA-containing oil” (control 2). In these cases the antioxidative stability is able to be increased still further by additionally adding tocopherol.

TABLE 2
Peroxide values after open storage

| Storage time | Control 1 | Control 2 | Example 3 | Example 6 |
|---|---|---|---|---|
| 0 days | 0.5 | 0.7 | 0.6 | 0.6 |
| 2 days | 3.0 | 1.2 | 1.4 | 1.3 |
| 7 days | 9.5 | 3.0 | 1.9 | 2.7 |
| 14 days | 17.8 | 4.2 | 2.8 | 2.1 |
| 21 days | 74.5 | 24.9 | 3.1 | 4.2 |

### Determination of Antioxidative Capacity

The antioxidative capacity of controls 1 and 5 and also of example 5 was determined as follows:

### Method:

The samples were measured by the Photochem method. The Photochem® operates according to the photochemoluminescence (PCL) method. Using a photosensitizer, superoxide anion radicals are generated which are detected via their reaction with a chemoluminogenic substance (for example Luminol) and measurement of the resultant light. The more free radical traps (antioxidants) are present in the sample, the more strongly is the intensity of the photochemoluminescence attenuated in a concentration-dependent manner. The results are reported in equivalent Trolox concentration units. The instrument operates using standardized kits for measuring the integral antioxidative capacity of individual antioxidants and superoxide dismutase.

For determining the Trolox equivalents, the samples were diluted with n-hexane and used directly for the measurement.

The results obtained are summarized in table 3.

TABLE 3
Antioxidative capacity of some samples

| | Antioxidative capacity |
|---|---|
| Sample | Trolox equivalents (μg/ml) |
| Control 1 | 20.4 |
| Control 5 | 328 |
| Example 5 | 1143 |

It may be seen that the examples according to the invention have comparatively high antioxidative capacities. It must be noted in this context that the amount of the added extract is not equivalent to the amount of antioxidatively active amount in the mixture. For instance, further purifications are possible and lead to extracts which are still more antioxidatively active. Of course, the scope of protection covers still more highly purified concentrates up to the antioxidatively active compounds.

1. A method for an antioxidative stabilization of fatty acid compositions comprising the steps of:
providing a fatty acid composition;
adding an extract from Crypthecodinium sp. having antioxidative activity.

2. The method of claim 1, wherein the extract has an antioxidative capacity of greater than 25 000 Trolox equivalents.

3. The method of claim 2, characterized in that the extract is obtainable by
i) saponifying a biomass of Crypthecodinium sp. and
ii) extracting the saponified biomass with a solvent which has a water solubility less than 0.1 g of solvent per g of water at 25° C.

4. The method of claim 3, characterized in that the extract is obtainable by extracting a saponified biomass from Crypthecodinium sp. with a substance selected from the group: hexane, pentane, ethyl acetate, diethyl ether, dichloromethane, dimethyl ethyl ketone, supercritical carbon dioxide, or combinations thereof.

5. The method of claim 2, characterized in that the extract is obtainable by extracting a biomass of Crypthecodinium sp. with an alcohol having 1 to 12 carbon atoms and/or with a ketone having 3 to 6 carbon atoms.

6. The method of claim 5, characterized in that the extract is obtainable by extracting a biomass of Crypthecodinium sp. with methanol, isopropanol, acetone and/or ethanol.

7. The method of claim characterized in that the extract is obtainable by extracting a biomass of Crypthecodinium sp. with an alcohol having 1 to 12 carbon atoms and subsequent extraction with a ketone.

8. The method of claim 1, characterized in that the extract is an extract from Crypthecodinium cohnii.

9. The method of claim 1, characterized in that the extract is obtainable by a method in which a biomass of Crypthecodinium sp. is transesterified.

10. The method of claim 1, characterized in that the extract is obtainable by a method in which a biomass of Crypthecodinium sp. is mechanically extracted.

11. An antioxidatively stabilized fatty acid composition which contains at least one unsaturated fatty acid, characterized in that the fatty acid composition contains at least one extract from Crypthecodinium sp. having antioxidative activity and components of a biomass different from Crypthecodinium sp.

12. The antioxidatively stabilized fatty acid composition as claimed in claim 11, characterized in that it contains components of a biomass of Thraustochytriales.

13. The antioxidatively stabilized fatty acid composition as claimed in claim 12, characterized in that it contains components of a biomass of Ulkenia sp.

14. The antioxidatively stabilized fatty acid composition as claimed in claim 11, characterized in that the components of the biomass different from Crypthecodinium sp. are obtainable by a method in which a biomass different from Crypthecodinium sp. is transesterified.

15. The antioxidatively stabilized fatty acid composition as claimed in claim 11, characterized in that it, in each case based on the total weight of the fatty acid composition, contains 0.1 to 50.0% by weight of at least one extract as claimed in claim 1 and 50.0 to 99.9% by weight of components of a biomass different from Crypthecodinium sp.

16. The antioxidatively stabilized fatty acid composition as claimed in claim 11, characterized in that it, based on its total weight, contains at least 25.0% by weight docosahexaenoic acid and/or docosahexaenoic acid alkyl ester.

17. The antioxidatively stabilized fatty acid composition as claimed in claim 11, characterized in that it contains at least one antioxidant.

18. The antioxidatively stabilized fatty acid composition as claimed in claim 17, characterized in that it contains α-, β-, γ- and/or δ-tocopherol and/or at least one tocotrienol.

19. The antioxidatively stabilized fatty acid composition as claimed in claim 18, characterized in that it in addition contains ascorbyl palmitate.

20. A method for producing an antioxidatively stabilized fatty acid composition comprising the steps of:
providing at least one extract from Crypthecodinium sp. having antioxidative activity;
providing components of a biomass different from Crypthecodinium sp.; and
mixing said extract from Crypthecodinium sp. with said components of a biomass different from Crypthecodinium sp.

21. The fatty acid composition as claimed in claim wherein said fatty acid composition is used as an active ingredient or component in pharmaceutical compositions.

22. The fatty acid composition as claimed in claim 11 wherein said fatty acid composition is used as a component in cosmetics preparations.

23. The use of a fatty acid composition as claimed in claim 11 wherein said fatty acid composition is used as a food additive and/or as food ingredient.

24. The fatty acid composition as claimed in claim 11 wherein said fatty acid composition is used as a component of animal feed.
